(19) United States
(12) Patent Application Publication
(10) Pub. No.: US 2026/0272378 A1
(43) Pub. Date: Sep. 17, 2026

(54) MUSCLE MASS MEASUREMENT SYSTEM AND MUSCLE MASS MEASUREMENT METHOD USING SAME

(71) Applicant: AJOU UNIVERSITY INDUSTRY-ACADEMIC COOPERATION FOUNDATION, Suwon-si, Gyeonggi-do (KR)

(72) Inventor: Sung Hyeon NOH, Seoul (KR)

(73) Assignee: AJOU UNIVERSITY INDUSTRY-ACADEMIC COOPERATION FOUNDATION, Suwon-si, Gyeonggi-do (KR)

(21) Appl. No.: 19/124,965

(22) PCT Filed: Oct. 25, 2023
(86) PCT No.: PCT/KR2023/016668

§ 371 Date: May 14, 2026

(30) Foreign Application Priority Data
Oct. 27, 2022 (KR) 10-2022-0139881

(51) Int. Cl. A61B 5/00; A61B 5/055

(52) U.S. Cl. CPC A61B 5/4872; A61B 5/055; A61B 5/4519

(57) ABSTRACT

The present invention relates to a system for measuring a muscle mass of a specific muscle. The system receives a plurality of MRI (Magnetic Resonance Imaging) tomographic images acquired by scanning along a single axis of a muscle to be measured, calculates a location, contour, and fat fraction of the muscle to be measured based on the images, and ultimately calculates a volume excluding fat of the muscle to be measured.

Accordingly, the time required for measuring muscle mass can be reduced, and a measurement result close to an actual value can be obtained.

### TECHNICAL FIELD

The present invention relates to a measuring system of muscle mass and measurement method of muscle mass using the same, and more particularly, to a muscle mass measurement system capable of reducing the measurement time and obtaining measurement results closer to actual values, as well as a muscle mass measurement method using the same.

### BACKGROUND

Muscle refers to a bodily organ composed of muscle cells, and performs body movement, protects internal organs, and assists in functions such as blood circulation and digestion. Specifically, it is involved in physical activities such as maintaining physique and walking, as well as in energy homeostasis, heat generation, and metabolism of sugar and amino acids.

Muscle accounts for nearly half of body weight, and in cases where muscle is deficient, there is a possibility of increased vulnerability to fractures and degenerative diseases. In particular, in the case of degenerative diseases, muscle mass is one of the most influential factors in the onset or prevention thereof.

Accordingly, for the diagnosis and prevention of various diseases including degenerative diseases, it may be required to measure muscle mass and changes thereof. However, there are limitations in measuring muscle mass values that are close to actual values.

Taking paraspinal muscle as an example, conventional paraspinal muscle mass measurement systems estimate paraspinal muscle mass based on cross-sectional images of the third and fourth lumbar vertebrae. However, in such types of muscle mass measurement systems, an expert individually observes each image and estimates muscle mass therefrom, and thus, there is a possibility that the measurement time may be excessively long. Furthermore, since only cross-sections of specific portions of the spine are referred to, there is difficulty in accurately measuring muscle mass.

Accordingly, development of a muscle mass measurement system capable of reducing measurement time and enabling muscle mass measurement close to actual values may be considered.

Korean Registered Patent Gazette No. 10-2396269 discloses a method for providing information necessary for diagnosis of CMT type 1A through quantitative measurement of thigh muscle. Specifically, it discloses a method for quantitatively measuring thigh muscle using MRI and providing information necessary for diagnosis of CMT type 1A based thereon.

However, in this type of measurement method, thigh muscle mass is estimated based only on cross-sectional images of the upper one-third point and the lower one-third point of the thigh, and thus, there is a possibility that a large difference may occur between the estimated muscle mass and the actual muscle mass.

Korean Patent Publication No. 2003-0090082 discloses a method for calculating volume of an extraction target using computer image information. Specifically, it discloses a method for extracting the volume of a specific organ from an image of an organ acquired by CT.

However, in this type of volume calculation method, since the image of the organ to be extracted is still output as a two-dimensional cross-section, there is a limitation in accurately calculating the volume. In addition, it is required that separate information be input by a user.
(Patent Document 1) Korean Registered Patent Gazette No. 10-2396269 (May 10, 2022)
(Patent Document 2) Korean Patent Publication No. 2003-0090082 (November 28, 2003)

### SUMMARY OF THE INVENTION

### Technical Problem

An object of the present invention is to provide a measuring system of muscle mass and a measurement method of muscle mass using the same, in which the muscle mass measurement time can be more reduced.

Another object of the present invention is to provide a measuring system of muscle mass and a measurement method of muscle mass using the same, which can obtain measurement results closer to actual values.

The problems of the present invention are not limited to those mentioned above, and other problems not mentioned will be clearly understood by those of ordinary skill in the art from the following description.

### Technical Solution

In order to achieve the above objects, a measuring system of muscle mass according to one aspect of the present invention is a system for measuring a muscle mass of a specific muscle, may include: an input unit configured to receive a plurality of MRI (Magnetic Resonance Imaging) tomographic images acquired by scanning along a single axis of a muscle to be measured; a position extraction unit configured to estimate a location of the muscle to be measured based on the plurality of images received by the input unit; a contour generation unit configured to set a boundary line of the muscle to be measured based on the estimation result of the position extraction unit; a fat fraction calculation unit configured to calculate a fat fraction present inside the boundary line set by the contour generation unit; and a muscle mass calculation unit configured to calculate a volume of the muscle to be measured, excluding fat, based on the boundary line set by the contour generation unit and a calculation result of the fat fraction calculation unit for the plurality of images received by the input unit.

In this case, the input unit is configured to receive an imaging position interval between each of the plurality of images received by the input unit, and the muscle mass calculation unit is configured to sequentially arrange the plurality of images so as to correspond to the imaging position interval received by the input unit, and to estimate a three-dimensional shape of the muscle to be measured based on the arrangement.

In this case, the fat fraction calculation unit may recognize, as fat, a point having relatively high brightness compared to other points within the boundary range set by the contour generation unit.

In this case, the muscle to be measured may be a paraspinal muscle.

In this case, the position extraction unit may estimate positions of a multifidus muscle, an erector spinae muscle, a psoas muscle, and a quadratus lumborum, respectively, based on the plurality of images received by the input unit, and the contour generation unit may set boundary lines of the multifidus muscle, the erector spinae muscle, the psoas muscle, and the quadratus lumborum, respectively, based on the estimation result of the position extraction unit.

In this case, the muscle mass calculation unit may calculate a volume excluding fat for each of the multifidus muscle, the erector spinae muscle, the psoas muscle, and the quadratus lumborum.

In this case, the present invention provides a measurement method of muscle mass for measuring a muscle mass of a specific muscle, the method comprising: (a) an input step in which a plurality of MRI (Magnetic Resonance Imaging) tomographic images acquired by scanning along a single axis of a muscle to be measured are input; (b) a position estimation step in which a location of the muscle to be measured is estimated based on the plurality of images; (c) a contour setting step in which a boundary line of the muscle to be measured is set based on the position ESTIMATION RESULT;
(d) a fat fraction calculation step in which a fat fraction present inside the boundary line is calculated; and (e) a muscle volume calculation step in which a volume of the muscle to be measured, excluding fat, is calculated based on the boundary line and the fat fraction calculation result with respect to the plurality of images.

In this case, the step (a) may include: (a1) a step in which a plurality of MRI (Magnetic Resonance Imaging) tomographic images acquired by scanning along a single axis of a paraspinal muscle are input.

In this case, the step (b) may include: (b1) a step in which positions of a multifidus muscle, an erector spinae muscle, a psoas muscle, and a quadratus lumborum are estimated, and the step (c) may include: (c1) a step in which boundary lines of the multifidus muscle, the erector spinae muscle, the psoas muscle, and the quadratus lumborum are set.

In this case, the step (e) may include: (e1) a step in which a volume excluding fat is calculated for each of the multifidus muscle, the erector spinae muscle, the psoas muscle, and the quadratus lumborum.

In this case, the step (d) may include: (d1) a step in which a point having relatively high brightness compared to other points within the boundary line is recognized as fat

In this case, the step (e) may include: (e2) a step in which the plurality of images are sequentially arranged so as to correspond to imaging position intervals; and (e3) a step in which a three-dimensional shape of the muscle to be measured is estimated.

### Advantageous Effects

Among the various effects of the present invention, the effects that can be obtained through the above-described means for solving the problems are as follows.

First, the measuring system of muscle mass according to an embodiment of the present invention includes an input unit, a position extraction unit, a contour generation unit, a fat fraction calculation unit, and a muscle mass calculation unit.

The position extraction unit, the contour generation unit, and the fat fraction calculation unit calculate the location, boundary line, and fat fraction of a muscle to be measured based on a plurality of MRI (Magnetic Resonance Imaging) tomographic images received by the input unit. The muscle mass calculation unit calculates a volume of the muscle to be measured excluding fat, based on the calculated location, boundary line, and fat fraction of the muscle to be measured.

In this case, the above-described calculation process is automatically performed according to preset rules. Therefore, compared to manual measurement, the time required to measure muscle mass can be more reduced.

In addition, the input unit receives a plurality of MRI tomographic images acquired by scanning along a single axis of a muscle to be measured, and the data calculation unit estimates the location, contour, and fat fraction of the muscle based on the received plurality of images.

Accordingly, muscle mass close to an actual value can be measured. As a result, the measurement accuracy can be improved.

Advantageous effects of the present invention are not limited to the above-described effects, and should be understood to include all effects that can be inferred from the configuration of the invention described in the description or claims of the present invention.

### BRIEF DESCRIPTION OF THE DRAWINGS

FIG. 1 is a schematic diagram illustrating a measuring system of muscle mass according to the first embodiment of the present invention.

FIG. 2 to 3 are conceptual diagram illustrating a process of extracting a location of a muscle to be measured and generating a contour using the measuring system of muscle mass of FIG. 1.

FIG. 4 is a flowchart illustrating a measurement method of muscle mass according to the first embodiment of the present invention.

FIG. 5 is a flowchart illustrating a measurement method of muscle mass according to the second embodiment of the present invention.

### DETAILED DESCRIPTION OF THE EMBODIMENTS

Hereinafter, a measuring system of muscle mass 1 according to the first embodiment of the present invention and a measurement method of muscle mass using the same will be described in more detail with reference to the drawings.

In the following description, descriptions of some components may be omitted in order to clearly illustrate the features of the present invention.

In the present specification, the same reference numerals are assigned to the same components, even in different embodiments, and repeated descriptions thereof will be omitted.

The accompanying drawings are provided only to facilitate the understanding of the embodiments disclosed in the present specification, and the technical spirit disclosed herein is not limited by the drawings.

Singular expressions include plural expressions unless clearly indicated otherwise by the context.

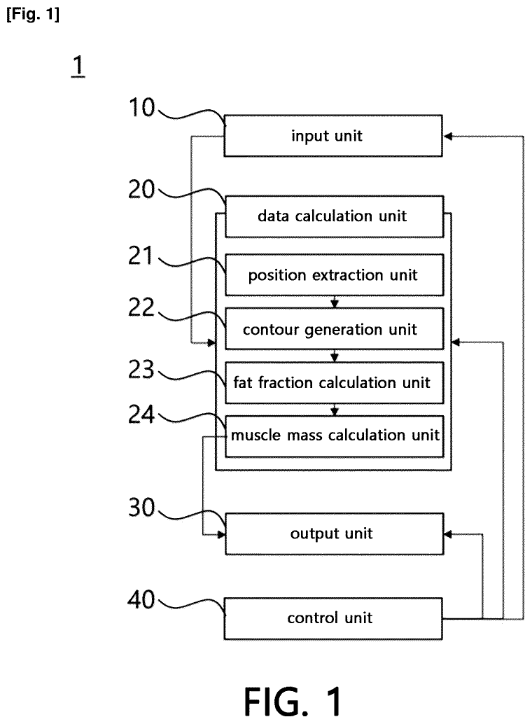

Hereinafter, a measuring system of muscle mass 1 according to the embodiment of the present invention will be described with reference to FIGS. 1 to 3.

The measuring system of muscle mass 1 according to the embodiment of the present invention measures a volume excluding fat of a specific muscle. In the first embodiment, the measuring system of muscle mass 1 may measure a volume excluding fat of a paraspinal muscle.

In the illustrated embodiment, the measuring system of muscle mass 1 includes an input unit 10, a data calculation unit 20, an output unit 30, and a control unit 40.

The input unit 10 receives MRI (Magnetic Resonance Imaging) tomographic images of a muscle to be measured. Specifically, the input unit 10 receives a plurality of MRI tomographic images acquired by scanning along a single axis of the muscle to be measured.

In addition, the input unit 10 may receive imaging position intervals of each of the plurality of images along with the plurality of images.

In the first embodiment, the input unit 10 may receive 40 MRI tomographic images of a paraspinal muscle, which are acquired at regular intervals by scanning the paraspinal muscle along a single axis using the MRI scanner.

The data received by the input unit 10 is delivered to the data calculation unit 20.

The data calculation unit 20 calculates a volume excluding fat of a muscle to be measured based of the data received by the input unit 10. Specifically, the data calculation unit 20 calculates a location, a contour, a fat fraction of the muscle to be measured, and calculates a volume excluding fat based thereon.

In this case, the data calculation unit 20 calculates a volume of the muscle to be measured by taking into account all of the plurality of MRI tomographic images received by the input unit 10. Accordingly, a three-dimensional shape of the muscle close to the actual shape can be estimated. Furthermore, muscle mass close to an actual value can be measured. As a result, the measurement accuracy can be further improved.

In the illustrated embodiment, the data calculation unit 20 includes a position extraction unit 21, a contour generation unit 22, a fat fraction calculation unit 23, and a muscle mass calculation unit 24.

The position extraction unit 21 is a component that estimates a location of the

### Muscle to Be Measured.

The position extraction unit 21 estimates the location of the muscle to be measured based on the plurality of MRI tomographic images received by the input unit 10.

In the first embodiment, the position extraction unit 21 may estimate a location of a paraspinal muscle based on a plurality of MRI tomographic images of the paraspinal muscle received by the input unit 10.

In the second embodiment, the position extraction unit 21 may estimate respective locations of a multifidus muscle, an erector spinae muscle, a psoas muscle, and a quadratus lumborum based on the plurality of MRI tomographic images of the paraspinal muscle received by the input unit 10.

The position extraction unit 21 delivers the estimation result to the contour generation unit 22.

The contour generation unit 22 sets a boundary line of the muscle to be measured based on the estimation result of the position extraction unit 21.

In the first embodiment, the contour generation unit 22 may set a boundary line of a paraspinal muscle based on the estimation result for the paraspinal muscle by the position extraction unit 21.

In the second embodiment, the contour generation unit 22 may set boundary lines of a multifidus muscle, an erector spinae muscle, a psoas muscle, and a quadratus lumborum, respectively, based on the estimation results for each of the muscles by the position extraction unit 21.

The position extraction unit 21 and the contour generation unit 22 may perform preprocessing and rescaling operations on the plurality of images received by the input unit 10.

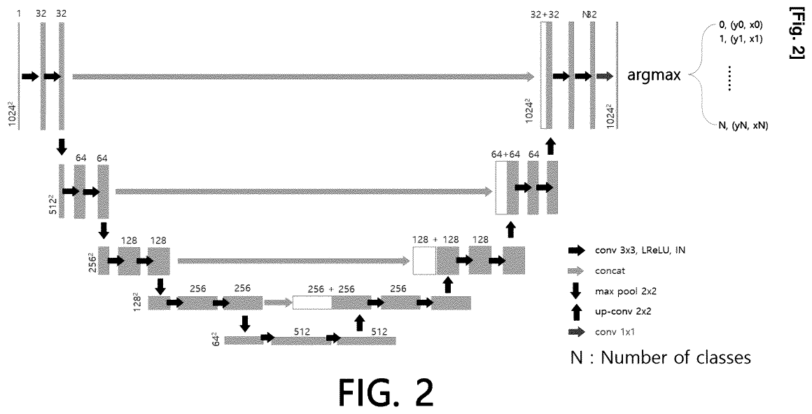

In addition, the position extraction unit 21 and the contour generation unit 22 may each be trained using deep learning based on an area detection model (see FIG. 2). However, the number of input data, scale, and the like for the position extraction unit 21 and the contour generation unit 22 may be arbitrarily adjusted according to setting conditions and environments.

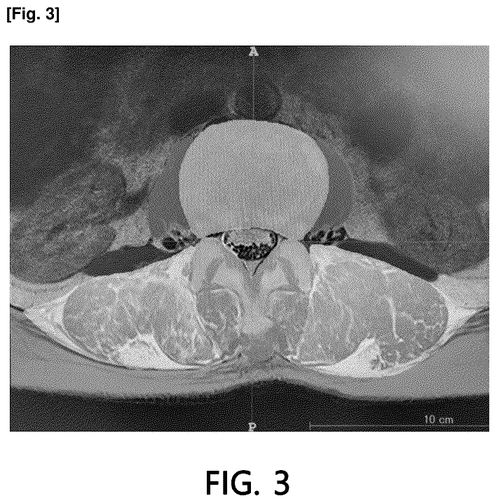

Referring to FIG. 3, an example of a result of setting a boundary line of the muscle to be measured for one MRI tomographic image can be confirmed. In the embodiment illustrated in FIG. 3, the green region, the yellow region, the red region, and the blue region represent contours of the multifidus muscle, the erector spinae muscle, the psoas muscle, and the quadratus lumborum, respectively. In addition, the light blue region and the pink region represent contours of the vertebra and fat, respectively.

Fat may also be included inside the boundary line set by the contour generation unit 22. To distinguish this, the fat fraction calculation unit 23 calculates a fat fraction inside the boundary line.

In the first embodiment, the fat fraction calculation unit 23 may recognize, as fat, a point having a relatively high brightness compared to other points within the boundary line set by the contour generation unit 22. In the second embodiment, the fat fraction calculation unit 23 may recognize, as fat, a point indicated in white within the boundary line set by the contour generation unit 22.

When the calculations of the location, contour, and fat fraction of the muscle to be measured are completed, the muscle mass calculation unit 24 calculates a volume excluding fat of the muscle to be measured based on the above-described calculation results.

The muscle mass calculation unit 24 calculates a volume excluding fat of the muscle to be measured based on the boundary line set by the contour generation unit 22 and the calculation result of the fat fraction calculation unit 23 for the plurality of images received by the input unit 10.

The muscle mass calculation unit 24 sequentially arranges the plurality of MRI tomographic images so as to correspond to imaging position intervals received by the input unit 10, and estimates a three-dimensional shape of the muscle to be measured based thereon.

In the first embodiment, the muscle mass calculation unit 24 may calculate a volume excluding fat for each of the multifidus muscle, the erector spinae muscle, the psoas muscle, and the quadratus lumborum.

The calculation operations of the position extraction unit 21, the contour generation unit 22, the fat fraction calculation unit 23, and the muscle mass calculation unit 24 are automatically performed according to preset rules. Therefore, compared to manual measurement, the time required for measuring muscle mass can be more reduced.

The result value of the muscle mass calculation unit 24 is delivered to the output unit 30.

The output unit 30 receives a volume value excluding fat of the muscle to be measured from the muscle mass calculation unit 24 and displays the value externally.

In the first embodiment, the output unit 30 may display the volume value excluding fat of the muscle to be measured externally through a display (not shown). In the second embodiment, the output unit 30 may transmit the volume value excluding fat of the muscle to be measured to a service server, and a user may check the value through a terminal.

The operations of the input unit 10, the data calculation unit 20, and the output unit 30 described above may be controlled by the control unit 40.

The control unit 40 controls the overall operations including activation and deactivation of the input unit 10, the data calculation unit 20, and the output unit 30. To this end, the control unit 40 is electrically connected to the input unit 10, the data calculation unit 20, and the output unit 30 so as to supply power thereto.

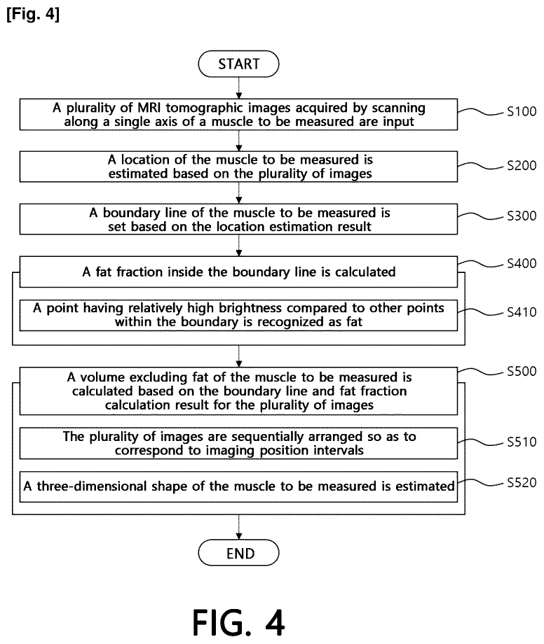

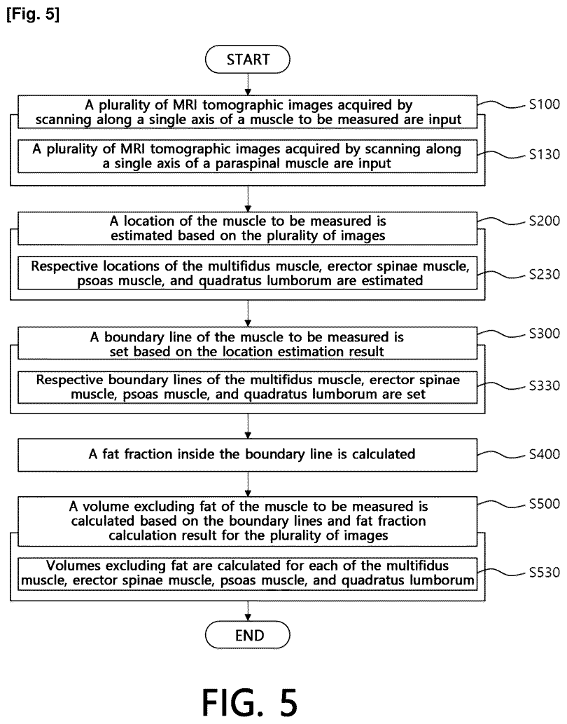

As described above, the measuring system of muscle mass 1 according to the embodiment of the present invention has been explained. Hereinafter, a measurement method of muscle mass according to the embodiment of the present invention will be described with reference to FIGS. 4 to 5.

FIG. 4 illustrates a measurement method of muscle mass according to the first embodiment of the present invention.

In the illustrated embodiment, the measurement method of muscle mass includes: a step (S100) in which a plurality of MRI tomographic images acquired by scanning along a single axis of the muscle to be measured are input; a step (S200) in which a location of the muscle to be measured is estimated based on the plurality of images; a step (S300) in which a boundary line of the muscle to be measured is set based on the result of the location estimation; a step (S400) in which a fat fraction inside the boundary line is calculated; and a step (S500) in which a volume excluding fat of the muscle to be measured is calculated based on the boundary line and the fat fraction calculation result for the plurality of images.

First, step (S100), in which a plurality of MRI tomographic images acquired by scanning along a single axis of the muscle to be measured are input, will be described.

The input unit 10 receives a plurality of MRI tomographic images of the muscle to be measured. In this case, the input unit 10 may also receive information on the imaging position intervals of the plurality of MRI tomographic images.

Next, step (S200), in which a location of the muscle to be measured is estimated based on the plurality of images, is performed.

The position extraction unit 21 estimates a location of the muscle to be measured based on the plurality of images received by the input unit 10.

In the first embodiment, the position extraction unit 21 may perform preprocessing and rescaling operations on the plurality of images.

In the second embodiment, the position extraction unit 21 may be trained using deep learning based on an area detection model.

Thereafter, step (S300), in which a boundary line of the muscle to be measured is set based on the result of the location estimation, is performed.

The contour generation unit 22 sets a boundary line of the muscle to be measured based on the estimation result of the position extraction unit 21.

In the first embodiment, the contour generation unit 22 may perform preprocessing and rescaling operations on the plurality of images and the corresponding estimation results from the position extraction unit 21.

In the second embodiment, the contour generation unit 22 may be trained using deep learning based on an area detection model.

Next, step (S400), in which a fat fraction inside the boundary line is calculated, is performed.

In the first embodiment, step (S400), in which a fat fraction inside the boundary line is calculated, may include step (S410), in which a point having relatively high brightness compared to other points within the boundary is recognized as fat.

When the calculation processes of the location, contour, and fat fraction of the muscle to be measured are completed, step (S500), in which a volume excluding fat of the muscle to be measured is calculated based on the boundary line and the fat fraction calculation result for the plurality of images, is performed.

In the first embodiment, step (S500), in which a volume excluding fat of the muscle to be measured is calculated based on the boundary line and the fat fraction calculation result for the plurality of images, may include: step (S510), in which the plurality of images are sequentially arranged so as to correspond to imaging position intervals; and step (S520), in which a three-dimensional shape of the muscle to be measured is estimated.

FIG. 5 illustrates a measurement method of muscle mass according to the second embodiment of the present invention.

The measurement method of muscle mass according to the present embodiment is intended to measure a volume excluding fat of a paraspinal muscle. The measurement method of muscle mass according to the present embodiment, like the measurement method of muscle mass illustrated in FIG. 4, includes: step (S100), in which a plurality of MRI tomographic images acquired by scanning along a single axis of the muscle to be measured are input; step (S200), in which a location of the muscle to be measured is estimated based on the plurality of images; step (S300), in which a boundary line of the muscle to be measured is set based on the result of the location estimation; step (S400), in which a fat fraction inside the boundary line is calculated; and step (S500), in which a volume excluding fat of the muscle to be measured is calculated based on the boundary line and the fat fraction calculation result for the plurality of images.

However, the measurement method of muscle mass according to the present embodiment differs from the above-described embodiment in that the paraspinal muscle is used as the muscle to be measured, and volumes of respective detailed muscles of the paraspinal muscle can be measured.

In the present embodiment, step (S100), in which a plurality of MRI tomographic images acquired by scanning along a single axis of the muscle to be measured are input, includes step (S130), in which a plurality of MRI tomographic images acquired by scanning along a single axis of a paraspinal muscle are input.

In addition, step (S200), in which a location of the muscle to be measured is estimated based on the plurality of images, and step (S300), in which a boundary line of the muscle to be measured is set based on the estimation result, include: step (S230), in which locations of a multifidus muscle, an erector spinae muscle, a psoas muscle, and a quadratus lumborum are estimated, respectively; and step (S330), in which boundary lines of the multifidus muscle, the erector spinae muscle, the psoas muscle, and the quadratus lumborum are set, respectively.

When the operations for estimating locations and setting boundary lines of the multifidus muscle, the erector spinae muscle, the psoas muscle, and the quadratus lumborum are completed, step (S400), in which a fat fraction inside the boundary lines is calculated, and step (S500), in which a volume excluding fat of the muscle to be measured is calculated based on the boundary lines and the fat fraction calculation result for the plurality of images, are performed.

In this case, step (S500), in which a volume excluding fat of the muscle to be measured is calculated based on the boundary lines and the fat fraction calculation results for the plurality of images, includes step (S530), in which volumes excluding fat are calculated for each of the multifidus muscle, the erector spinae muscle, the psoas muscle, and the quadratus lumborum.

While the preferred embodiments of the present invention have been described above with reference to specific examples, the present invention is not limited to the configurations of the described embodiments.

Furthermore, various modifications and changes may be made by those skilled in the art without departing from the spirit and scope of the present invention as defined in the claims.

Moreover, the above embodiments may be selectively combined in whole or in part so as to implement various modifications.

1. A system for measuring a muscle mass of a specific muscle, comprising:
an input unit configured to receive a plurality of MRI (Magnetic Resonance Imaging) tomographic images acquired by scanning along a single axis of a muscle to be measured;
a position extraction unit configured to estimate a location of the muscle to be measured based on the plurality of images received by the input unit;
a contour generation unit configured to set a boundary line of the muscle to be measured based on the estimation result of the position extraction unit;
a fat fraction calculation unit configured to calculate a fat fraction inside the boundary line set by the contour generation unit; and
a muscle mass calculation unit configured to calculate a volume excluding fat of the muscle to be measured based on the boundary line set by the contour generation unit and the calculation result of the fat fraction calculation unit for the plurality of images received by the input unit.

2. The system of claim 1, wherein the input unit is further configured to receive imaging position intervals of the plurality of images,
and the muscle mass calculation unit is further configured to sequentially arrange the plurality of images so as to correspond to the imaging position intervals received by the input unit, and estimate a three-dimensional shape of the muscle to be measured based thereon.

3. The system of claim 1, wherein the fat fraction calculation unit is configured to recognize, as fat, a point having relatively high brightness compared to other points within the boundary line set by the contour generation unit.

4. The system of claim 1, wherein the muscle to be measured is a paraspinal muscle.

5. The system of claim 4, wherein the position extraction unit is configured to estimate respective locations of a multifidus muscle, an erector spinae muscle, a psoas muscle, and a quadratus lumborum based on the plurality of images received by the input unit,
and the contour generation unit is configured to set respective boundary lines of the multifidus muscle, the erector spinae muscle, the psoas muscle, and the quadratus lumborum based on the estimation result of the position extraction unit.

6. The system of claim 5, wherein the muscle mass calculation unit is configured to calculate a volume excluding fat for each of the multifidus muscle, the erector spinae muscle, the psoas muscle, and the quadratus lumborum.

7. A method for measuring a muscle mass of a specific muscle, comprising:
(a) inputting a plurality of MRI tomographic images acquired by scanning along a single axis of a muscle to be measured;
(b) estimating a location of the muscle to be measured based on the plurality of images;
(c) setting a boundary line of the muscle to be measured based on the result of the location estimation;
(d) calculating a fat fraction inside the boundary line; and
(e) calculating a volume excluding fat of the muscle to be measured based on the boundary line and the fat fraction calculation result for the plurality of images.

8. The method of claim 7, wherein step (a) includes:
(a1) inputting a plurality of MRI tomographic images acquired by scanning along a single axis of a paraspinal muscle.

9. The method of claim 8, wherein step (b) includes:
(b1) estimating respective locations of a multifidus muscle, an erector spinae muscle, a psoas muscle, and a quadratus lumborum; and
wherein step (c) includes:
(c1) setting respective boundary lines of the multifidus muscle, the erector spinae muscle, the psoas muscle, and the quadratus lumborum.

10. The method of claim 9, wherein step (e) includes:
(e1) calculating volumes excluding fat for each of the multifidus muscle, the erector spinae muscle, the psoas muscle, and the quadratus lumborum.

11. The method of claim 7, wherein step (d) includes:
(d1) recognizing, as fat, a point having relatively high brightness compared to other points within the boundary.

12. The method of claim 7, wherein step (e) includes:
(e2) sequentially arranging the plurality of images so as to correspond to imaging position intervals; and
(e3) estimating a three-dimensional shape of the muscle to be measured based thereon.
